(19) United States
(12) Patent Application Publication
(10) Pub. No.: US 2016/0074066 A1
(43) Pub. Date: Mar. 17, 2016

(54) TROCAR FOR THORACOSCOPIC SURGERY, AND PORT FORMATION KIT FOR THORACOSCOPIC SURGICAL INSTRUMENTS

(71) Applicant: Toshiaki MORIKAWA, Tokyo (JP)

(72) Inventor: Toshiaki MORIKAWA, Tokyo (JP)

(21) Appl. No.: 14/953,610

(22) Filed: Nov. 30, 2015

Related U.S. Application Data
(63) Continuation-in-part of application No. PCT/JP2014/064069, filed on May 28, 2014.

(30) Foreign Application Priority Data
May 31, 2013 (JP) 2013-115479
Dec. 26, 2013 (JP) 2013-269053

(51) Int. Cl. A61B 17/34; A61B 17/02

(52) U.S. Cl. CPC A61B 17/3423; A61B 17/0218; A61B 2017/3427; A61B 2017/3445

(57) ABSTRACT

A trocar for thoracoscopic surgery includes a cylindrical portion with an elliptical cross section, and an outer flange and an inner flange provided on respective ones of both ends of the cylindrical portion. The cylindrical portion has two planar side wall portions, two arcuate portions connecting the side wall portions at both ends thereof, and a partitioning wall portion partitioning the interior of the cylindrical portion between both side wall portions. Both side wall portions per se and both arcuate portions per se are formed in curved fashion in such a manner that outer surfaces thereof take on a concave shape.

### BACKGROUND OF THE INVENTION

1. Field of the Invention

This invention relates to a trocar for use in forming a port for inserting various instruments for thoracoscopic surgery (instruments such as a thoracoscope, forceps, retractor, aspirator and stapler) into the thoracic cavity, as well as a port formation kit that includes a trocar.

2. Description of the Related Art

A trocar or port formation kit (device), which is referred to as a surgical access device, access port and by other names, has been developed as an instrument for abdominal laparoscopic surgery for the purpose of facilitating the insertion of a laparoscope or the like while maintaining air tightness within the abdominal cavity and also protecting the incision. Basically, a trocar is composed of a cylindrical portion (sleeve) and a portion, referred to as a flange or ring, provided on both ends of the sleeve. For example, see Patent Documents 1 and 2.
Patent Document 1: Japanese Patent Application Laid-Open No. 2010-82450
Patent Document 2: Japanese Patent Application Laid-Open No. 2012-200608

These conventional trocars are premised on laparoscopic surgery and are not always suitable for thoracoscopic surgery the purpose of which is removal of a portion of the lung. In thoracoscopic surgery, an incision is made between ribs of the chest wall and the trocar is placed in the incision and adapted to somewhat forcibly spread the ribs apart. Since the trocar has a cylindrical portion, the ribs come into abutting point contact with this portion. Since force concentrates at this single point, the nerves innervating the periosteum on the rib surface are irritated, inflicting pain upon the patient. The pain remains after surgery.

### SUMMARY OF THE INVENTION

The present invention provides a trocar that is capable of alleviating, as much as possible, pain inflicted upon the patient, as well as a port formation kit.

Further, an object of the present invention is to enhance the operability of a surgical instrument inserted into the thoracic cavity.

A trocar for thoracoscopic surgery according to the present invention is for bringing into communication the inside and outside of the thoracic cavity which does not require that air tightness be maintained, the trocar being formed of a synthetic resin having such mechanical strength that the interior thereof will not collapse when the trocar is placed between the ribs. The trocar comprises a cylindrical portion with an elliptical cross section, and an outer flange and an inner flange provided on respective ones of both ends of the cylindrical portion; the cylindrical portion having two side wall portions extending in the same direction, arcuate portions connecting the side wall portions at both ends thereof, and a partitioning wall portion at least partially partitioning the interior of the cylindrical portion between both side wall portions; both side wall portions per se and both arcuate portions per se being formed in curved fashion in such a manner that outer surfaces thereof take on a concave shape from the outer flange to the inner flange. The two side wall portions are spaced apart and extend in the same direction, the arcuate portions being connected to respective ones of both ends of the two side wall portions. The cylindrical portion is formed by the two side wall portions and the two arcuate portions that connect respective ones of both edges thereof. Two openings (ports) are formed in the cylindrical portion by providing the partitioning wall portion for at least partially partitioning the interior of the cylindrical portion between both side wall portions. Three or more ports can be formed as well by providing two or more partitioning wall portions.

The trocar is placed between the ribs of the patient by being inserted into an incision formed along the ribs between the ribs. At least two ports are formed inside the cylindrical portion by the partitioning wall portion with which the cylindrical portion is provided, and thoracoscopic surgical instruments of various types can be inserted into the body of the patient through these ports. Since the outer surface is formed by being curved into a concave shape from the outer flange to the inner flange, namely along the entire cylindrical portion having the elliptical cross section, what comes into contact with the ribs is planar in shape and force applied to the ribs is dispersed. As a result, the patient's residual pain is alleviated. Further, in conformity with the shape of the slender space between the ribs, the cylindrical portion of the trocar likewise is somewhat slender (elliptical in cross section). Therefore, by providing the cylindrical portion with the partitioning wall portion, two or more ports can be acquired with use of a single trocar. Since the one end of the cylindrical portion that is on the outer side thereof is provided with a flange, the trocar is prevented from falling into the interior of the body of the patient.

Since both side wall portions per se and both arcuate portions per se are curved in such a manner that their outer surfaces take on a concave shape, the cylindrical portion spreads out toward the outer and inner flanges. Since a surgical instrument inserted from the side of the outer flange having such spread can be inserted into the port while traveling along the inner surface of the cylindrical portion, it is possible to insert the surgical instrument in a stable state (without wobble) and the orientation (angle of insertion) of the instrument can be changed as well. Accordingly, the operability of the surgical instrument inserted into the thoracic cavity is enhanced. Since the cylindrical portion (constituted by both side wall portions per se and both arcuate portions per se) is formed of synthetic resin having such mechanical strength that the cylindrical portion will not be crushed when placed between the ribs, the port through which the surgical instrument is passed will not be flattened by the ribs on either side thereof. Hence, the port through which the surgical instrument is passed in a state in which the trocar is placed between the ribs can be maintained at all time.

In a preferred embodiment, the cylindrical portion is inclined in such a direction that the dimensions thereof increase from the inner side toward the outer side. As a result, the trocar placed between the ribs is retained stably under an outwardly directed force.

Preferably, the outer flange and the inner flange project laterally from the cylindrical portion and are provided along the entire periphery of the cylindrical portion.

In a preferred embodiment, one end portion of the inner flange is formed tapering to a point. This facilitates insertion of the trocar into the incision of the thoracic cavity.

The partitioning wall portion between both side wall portions may be provided extending in a direction from the outer side to the inner side of the cylindrical portion up to a point or may be provided extending from the outer side to the inner side of the cylindrical portion. Further, the partitioning wall portion may be provided in a form bridging both side wall portions, or the partitioning wall may be provided with a gap. For example, the partitioning wall portion may be composed of a pair of projections each provided on the inner surface of a respective one of both side wall portions.

A port formation kit for thoracoscopic surgical instruments according to the present invention comprises a trocar and a spacer. The trocar includes a cylindrical portion with an elliptical cross section, and an outer flange and an inner flange provided on respective ones of both ends of the cylindrical portion; the cylindrical portion having two side wall portions extending in the same direction, arcuate portions connecting the side wall portions at both ends thereof, and at least one partitioning wall portion connecting both side wall portions. The spacer is in the shape of a U along the outer side of the cylindrical portion of the trocar. In a preferred embodiment, a hook is formed on a tip of the spacer. Preferably, the kit includes multiple spacers each of which has a different thickness.

If a spacer having a thickness conforming to a patient having a thin thoracic wall is inserted between the thoracic wall and the outer flange of the trocar, the trocar will be firmly fixed in place.

Other features and advantages of the present invention will be apparent from the following description taken in conjunction with the accompanying drawings, in which like reference characters designate the same or similar parts throughout the figures thereof.

### BRIEF DESCRIPTION OF THE DRAWINGS

FIG. 1 is a perspective view of a trocar for thoracoscopic surgery according to a first embodiment of the present invention;

FIG. 2 is a plan view of the trocar for thoracoscopic surgery according to the first embodiment;

FIG. 3 is a sectional view taken along line III-III of FIG. 2;

FIG. 4 is a sectional view taken along line IV-IV of FIG. 2;

FIG. 5 is a sectional view taken along line V-V of FIG. 3;

FIG. 6 is a perspective view illustrating a modification of the first embodiment;

FIG. 7A is a sectional view, which corresponds to FIG. 3, illustrating the modification of the first embodiment;

FIG. 7B illustrates how surgical instruments have been inserted into the trocar shown in FIG. 7A;

FIG. 8A is a sectional view, which corresponds to FIG. 4, illustrating the modification of the first embodiment;

FIG. 8B illustrates how a surgical instrument has been inserted into the trocar shown in FIG. 8A;

FIG. 9 is a perspective view illustrating another modification of the first embodiment;

FIG. 10 is a plan view, which corresponds to FIG. 2, illustrating a further modification of the first embodiment;

FIG. 11 is a plan view, which corresponds to FIG. 3, illustrating a further modification of the first embodiment;

FIG. 12 is a perspective view of a spacer;

FIG. 13 illustrates the manner in which the trocar of the first embodiment is used in thoracoscopic surgery;

FIG. 14 is a sectional view illustrating state of utilization of a spacer;

FIG. 15 is a perspective view of a trocar for thoracoscopic surgery according to a second embodiment of the present invention;

FIG. 16 is a sectional view, which corresponds to FIG. 3, illustrating the second embodiment;

FIG. 17 is a perspective view of a trocar for thoracoscopic surgery according to a modification of the second embodiment;

FIG. 18 is a plan view of a trocar for thoracoscopic surgery according to the modification of the second embodiment;

FIG. 19 is a sectional view taken along line XIX-XIX of FIG. 18;

FIG. 20 is a sectional view taken along line XX-XX of FIG. 18; and

FIG. 21 illustrates the manner in which the trocar of the second embodiment is used in thoracoscopic surgery.

### DESCRIPTION OF THE PREFERRED EMBODIMENTS

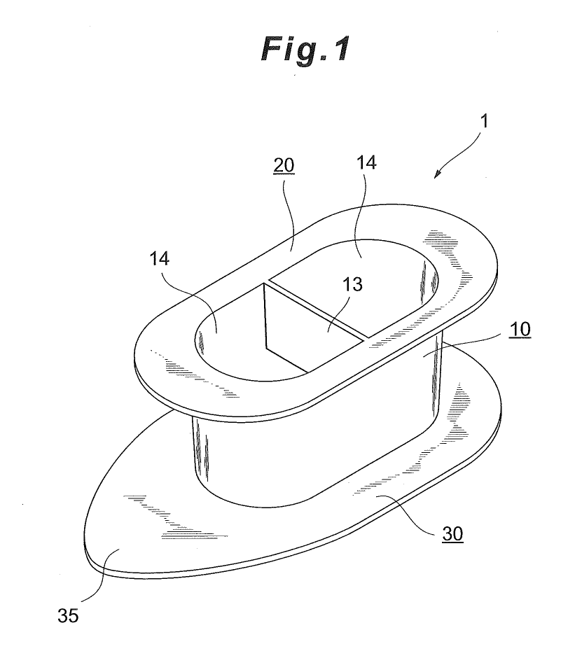

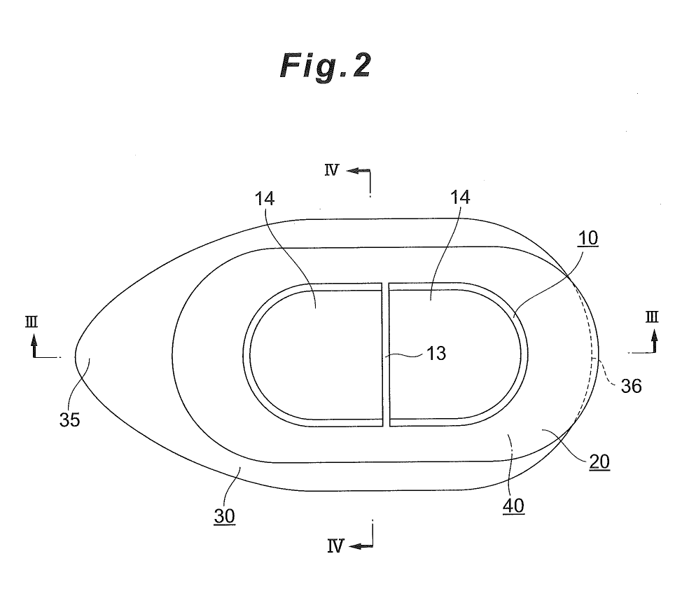

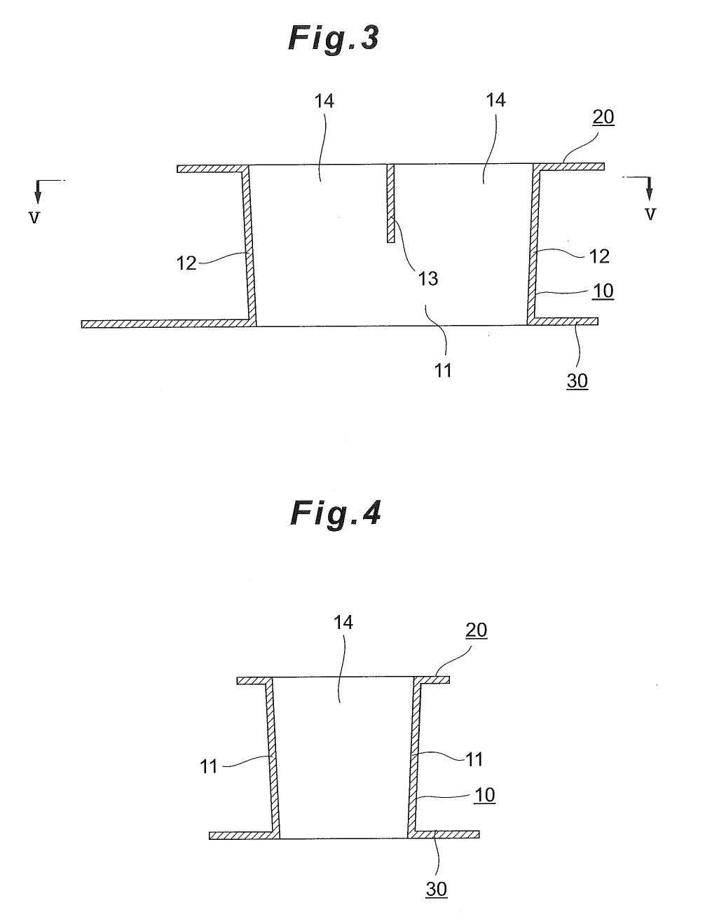

FIGS. 1 to 5 illustrate a trocar for thoracoscopic surgery according to a first embodiment of the present invention.

A trocar 1 for thoracoscopic surgery comprises a cylindrical portion 10 having an elliptical cross section, as well as an outer flange 20 and an inner flange 30 integrally provided on respective ones of both ends of the cylindrical portion 10. The ends of the cylindrical portion 10 with which the outer flange 20 and inner flange 30 are provided will be referred to as the outer side and inner side, respectively, of the cylindrical portion 10 or trocar.

The cylindrical portion 10 is constituted by two flat-plate-shaped side wall portions 11 of a certain (predetermined) length spaced apart and extending in the same direction (they need not be precisely parallel and slight curvature or inclination is allowable), and two arcuate portions 12 connecting both ends of the side wall portions 11 continuously and in arcuate fashion (preferably in semi-circular fashion). The cylindrical portion 10 is provided with a partitioning wall portion 13 connecting the two side wall portions 11 at the central portions thereof. The side wall portions 11, arcuate portions 12 and partitioning wall portion 13 have a thickness on the order of several millimeters, by way of example. By providing the partitioning wall portion 13, the interior of the cylindrical portion 10 is partitioned in two and two ports 14 are thus formed. The ports 14 are used to insert surgical instruments into the thoracic cavity. The partitioning wall portion 13 need not necessarily be situated centrally of the side wall portions 11 in the length direction thereof and the two ports 14 may have different sizes. If necessary, two or more side wall portions may be provided to form three or more ports. In the first embodiment, the partitioning wall portion 13 is provided extending from the outer side to the inner side of the cylindrical portion 10 up to a point midway between them. However, the partitioning wall portion 13 may be provided spanning the entire length of the cylindrical portion 10 (see FIG. 11).

The side wall portions 11 and arcuate portions 12 are inclined (they are not perpendicular to the flanges 20 and 30) in such a manner that the outer side of the cylindrical portion 10 is slightly larger than the inner side thereof overall. That is, the spacing between the two side wall portions 11 is slightly larger at the outer side of the cylindrical portion 10 than at the inner side thereof, and the spacing between the two arcuate portions 12 is slightly larger at the outer side of the cylindrical portion 10 than at the inner side thereof. The reason for this is that when the cylindrical portion 10 of the trocar 1 is inserted between ribs of the human body and is lodged between the ribs, the trocar 1 is retained stably at this time under an outwardly directed force. The size of the cylindrical portion 10 may of course be made uniform along the entirety thereof from the outer to the inner side. If the side wall portions of the cylindrical portion 10 are each formed to have a recess curved into a concave shape so as to conform to the roundness of the rib surface, rib contact will be planar rather than linear. Side wall portions having such recesses are intended to fall within the meaning of the term “planar” (“flat-plate-shaped”).

The outer flange 20 and inner flange 30 are parallel to each other and perpendicular to the center line of the cylindrical portion 10. The outer flange 20 and inner flange 30 also have a thickness on the order of several millimeters. The outer flange 20 follows the external shape of the side wall portions 11 and arcuate portions 12 of cylindrical portion 10, the portions thereof along the side wall portions 11 are of uniform width, and the portions thereof along the arcuate portions 12 are of somewhat greater width. The inner flange 30 also follows the external shape of the side wall portions 11 and arcuate portions 12 of cylindrical portion 10. The width of the portions of the inner flange 30 along the side wall portions 11 is uniform and somewhat greater than the corresponding portions of the outer flange 20. With regard to the portions of the inner flange 30 along the arcuate portions 12, one portion has a width the same as that of the portions along the side wall portions 11, and the other portion (indicated at reference numerals 35) tapers somewhat to a point (the tip of which is rounded slightly). When the trocar 1 is inserted into an incision formed in the thoracic wall, insertion is facilitated by first inserting the tip 35 of the inner flange 30. Conversely, the reason for making the flange portion (reference numerals 36) on the opposite side semicircular and reducing the width thereof (the width is substantially the same as that of the flange portions along the side wall portions 11) is to enable the trocar 1 to be removed easily when use of the trocar 1 ends.

The trocar 1 can be formed of a synthetic resin. For example, it is preferred that the trocar 1 be formed of a synthetic resin exhibiting high mechanical strength and good moldability (it will suffice if the synthetic resin has enough hardness to avoid flattening of the ports 14 when the trocar 1 is placed between the ribs), and that layers of a soft resin such as silicone rubber be built up on the inner surface (the inner surface of the cylindrical portion 10, the surface of the partitioning wall portion 13, the inner surface of the flange 20 and of the flange 30, etc.) or on the outer surface. The entire trocar 1 may be formed of a single layer or of multiple layers of synthetic resin. In any case, the ports 14 can be maintained at all times with the trocar in the inserted state without the ports 14 being flattened by the ribs.

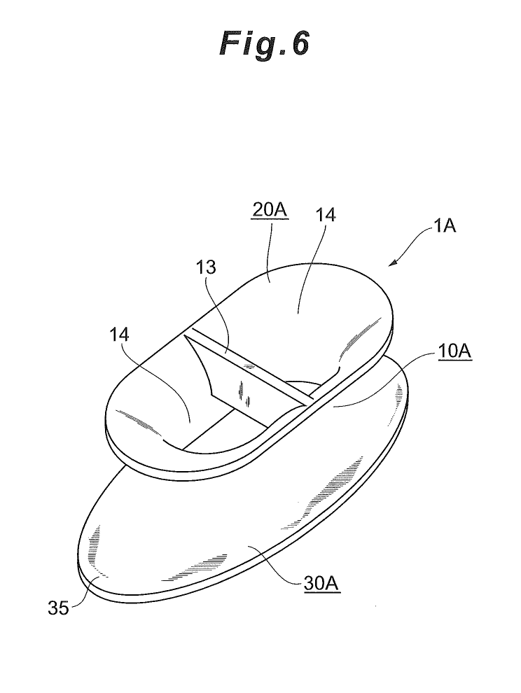

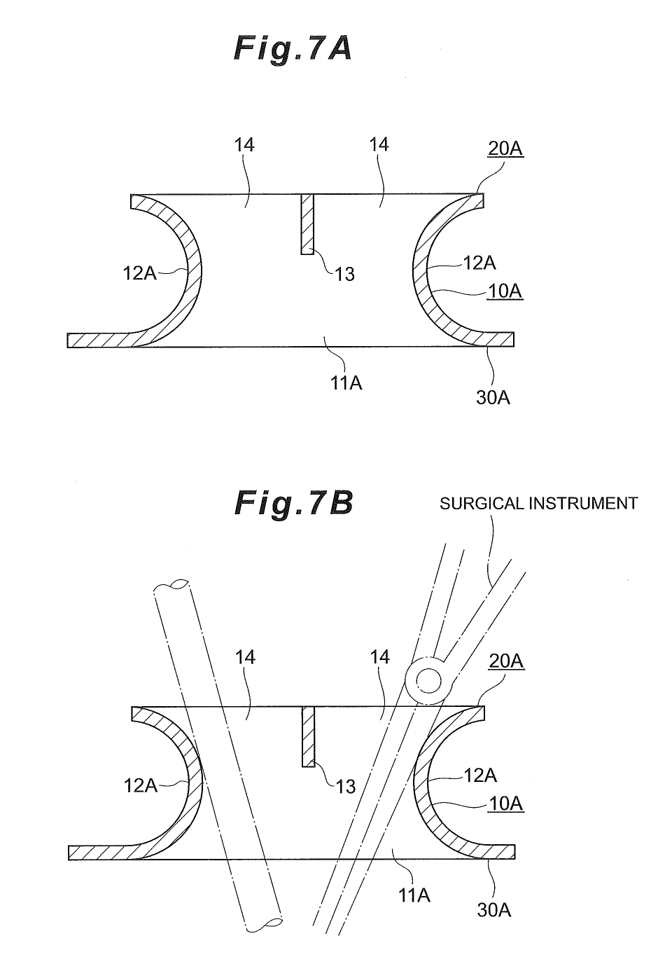

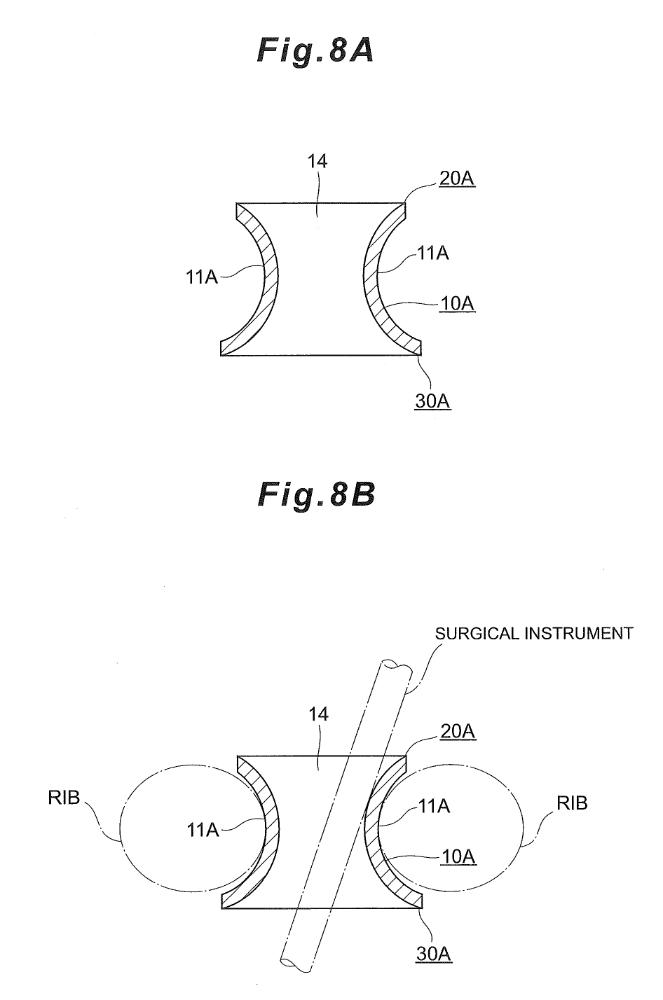

FIGS. 6 to 8B illustrate a modification of the trocar according to the first embodiment. In a trocar 1A of this modification, the outer surfaces of both side wall portions 11A per se of the cylindrical portion 10A are curved into a concave shape from an outer flange 20A to an inner flange 30A in such a manner that both side wall portions 11A of the cylindrical portion 10A will conform to the roundness of the rib surface. With regard also to both arcuate portions 12A per se of the cylindrical portion 10A, the outer surfaces thereof are curved into a concave shape from the outer flange 20A to the inner flange 30A. Since both side wall portions 11A per se and both arcuate portions 12A per se (the entire cylindrical portion 10A) are curved, their inner surfaces (namely the ports 14) also are curved into a concave shape from the outer flange 20A to the inner flange 30A in the same manner as the outer surfaces. That is, the cylindrical portion 10A widens gradually as it approaches the outer flange 20A and the inner flange 30A.

A sufficient clearance is assured between each port 14 of the trocar 1A and the surgical instrument that will be inserted into the port 14, allowing insertion and withdrawal of the surgical instrument as a matter of course and enabling the angle of the surgical instrument to be changed freely during surgery. The surgical instrument need not necessarily have a linear shape. For example, even forceps having a scissor-type configuration can be used. Since the trocar 1A is formed of synthetic resin the mechanical strength of which is such that the interior will not collapse when the trocar is placed between the ribs, the surgical instrument that will be inserted along the inner surface of the cylindrical portion 10 will be stabilized. Further, it is possible to change the angle of the surgical instrument after it is inserted, as mentioned above. Force applied to the surgical instrument will not be inflicted directly upon the ribs.

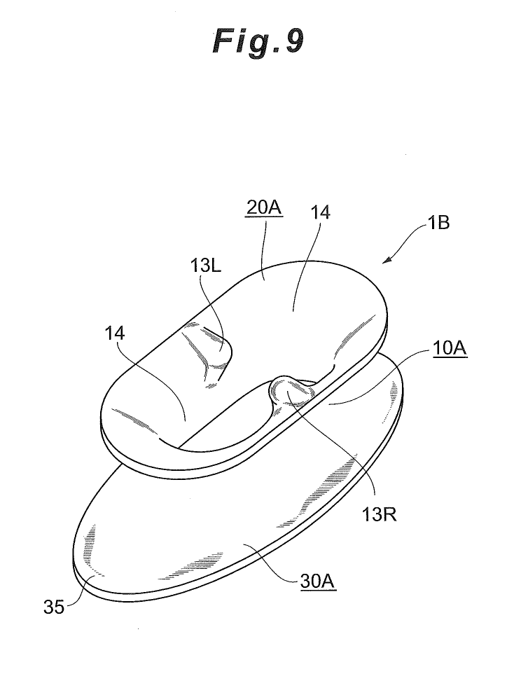

FIG. 9 illustrates another modification of the trocar according to the first embodiment. Although this trocar 1B has a basic shape the same as that of the trocar 1A described above, it differs in that the partitioning wall portion is constituted by a pair of projections 13L and 13R (the partitioning wall portion is formed to have a gap). The interior of the cylindrical portion 10A is partitioned approximately in two by the pair of projections 13L and 13R and two ports 14 are thus formed. By using the gap between the tips of the pair of projections 13L and 13R, a surgical instrument that has been inserted into either of the ports 14 can be moved a large amount (inclined by large amount). Further, a surgical instrument that has been inserted into one of the ports 14 can be moved to the other port 14 while remaining inserted in the first port 14.

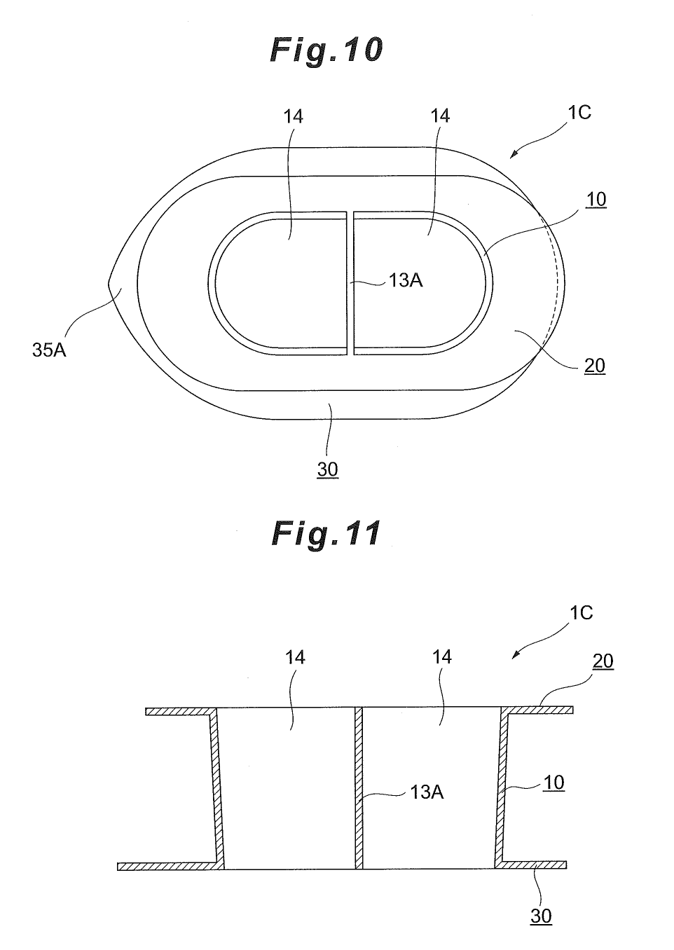

FIGS. 10 and 11 illustrate a further modification of the trocar according to the first embodiment. Here a trocar 1C is such that the length of tip 35A of the inner flange 30 is slightly smaller than that of the trocar 1 of the first embodiment set forth above. Further, partitioning wall portion 13A is provided spanning the entire length of the cylindrical portion 10. Thus, the shapes and sizes, etc., of the cylindrical portion, outer and inner flanges and partitioning wall portion as well as the number of partitioning wall portions, number and size, etc., of the ports are matters of design that can be decided appropriately.

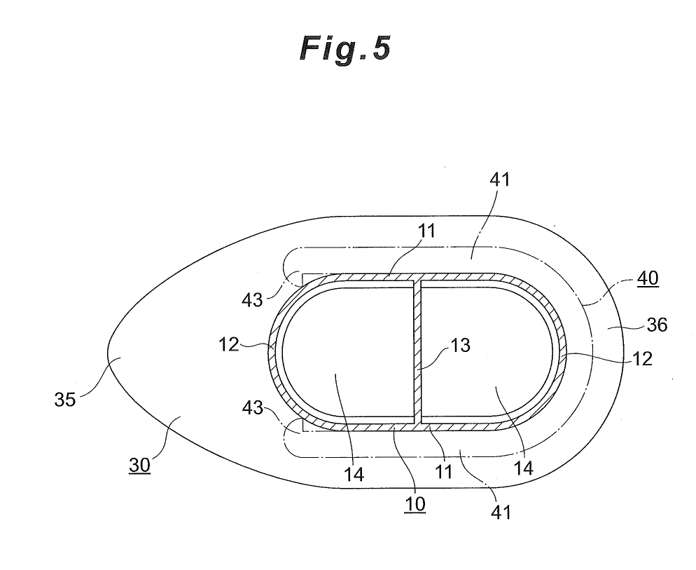

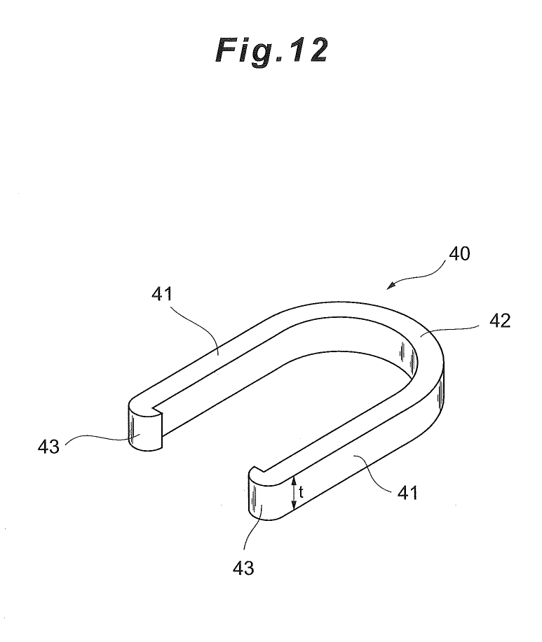

FIG. 12 illustrates an embodiment of a spacer 40. The spacer 40 is indicated by the phantom lines in FIG. 5 in relation to the trocar 1. The spacer 40, which has a U-shaped configuration overall, has a size conforming to the outer side of wall portions 11 and of one arcuate portion 12 of the cylindrical portion 10 of trocar 1. The spacer 40 also is formed of synthetic resin having suitable elasticity. The spacer 40 is constituted by two clamping portions 41 extending along the outer sides of the side wall portions 11 of the cylindrical portion 10 of trocar 1, and an arcuate connecting portion 42 connecting the clamping portions 41 at one end thereof. The tip of each of the clamping portions 41 is formed to have a hook 43. As shown in FIG. 5, the spacer 40 is attached to the outer side of the cylindrical portion 10 of trocar 1. The hooks 43 prevent the spacer 40 from coming off. Multiple spacers of different thickness t may be prepared in advance.

A port formation kit for thoracoscopic surgical instruments according to the present invention includes the above-described trocar and one spacer 40 or multiple spacers 40 of different thickness.

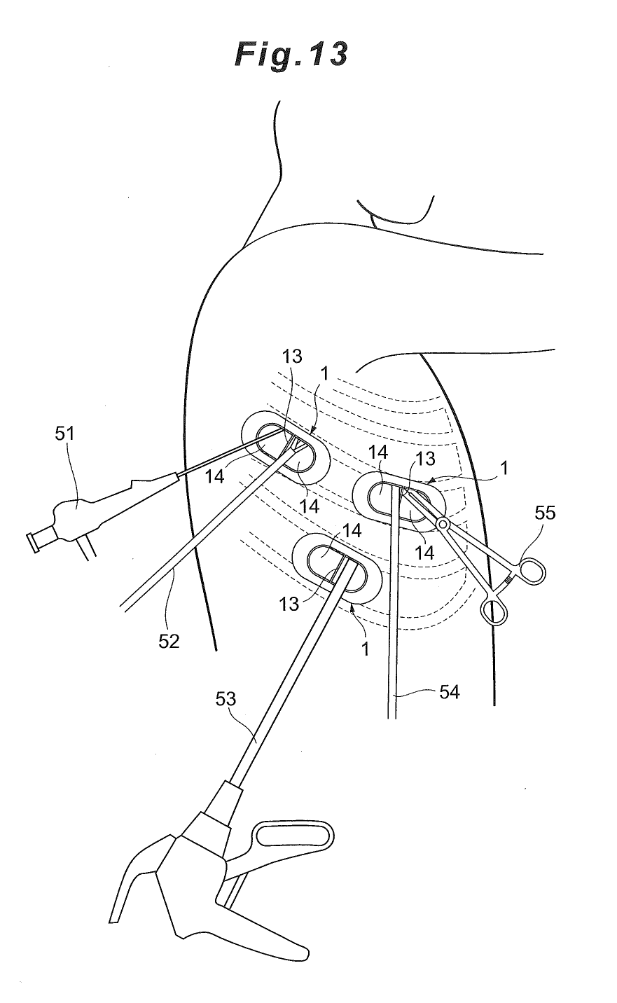

In thoracoscopic surgery, a suitable location on the patient's chest in incised between the ribs along the direction of the ribs, as shown in FIG. 13. The trocar 1 is inserted into the incision starting from the tip 35 of the inner flange 30. The side wall portions 11 of the cylindrical portion 10 of trocar 1 softly widen the incision and the inner flange 30 is inserted between the ribs. The inner flange 30 and outer flange 20 sandwich the thoracic wall of the patient from the inside and outside thereof between the two ribs. The cylindrical portion 10 of the trocar 1 is embraced by the two ribs on either side. Thus, the trocar 1 is passed through the incision and placed between the ribs of the thoracic wall and the port 14 is formed for bringing the inside and outside of the thoracic cavity into communication. The trocar 1 is placed at one or multiple locations as needed.

Since the trocar 1 is provided with two ports 14, surgical instruments such as a scope 51, retractor 52, stapler 53, aspirator 54 and forceps 55 can be inserted through these ports 14 to enable surgery and treatment. The trocar 1 is equipped with the cylindrical portion 10 of elliptical cross section having the two flat-plate-shaped side wall portions 11. Since the ribs of the patient come into abutting contact with the outer sides of the side wall portions 11 not at one point but in linear or planar fashion, force is dispersed and pain (especially post-operative pain) felt by the patient is alleviated. Further, the space between the ribs is band-shaped. Owing to the provision of the cylindrical portion 10 of elliptical cross section forming part of the trocar 1 having a longitudinal direction the same as that of this band-shaped portion, the band-shaped space between the ribs is utilized fully. Since the cylindrical portion of the trocar 1 has an elliptical cross section, two or more ports can be formed to permit the insertion of multiple surgical instruments, as mentioned above. It should be noted that thoracoscopic surgery does not require the thoracic cavity to be maintained air-tight as in laparoscopic surgery.

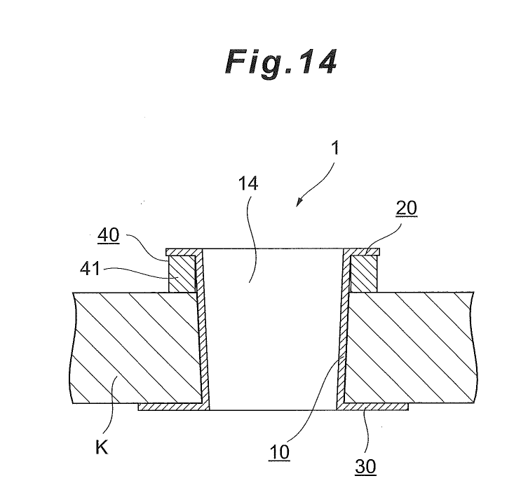

The thickness of the thoracic wall differs depending upon the patient. The spacer 40 preferably is used, as shown in FIG. 14, for a patient whose thoracic-wall thickness is smaller than the length of the cylindrical portion of the trocar. Specifically, if the spacer 40 of suitable thickness is placed between the outer flange 20 of the trocar 1 and thoracic wall K, the trocar 1 will be fixed firmly in place.

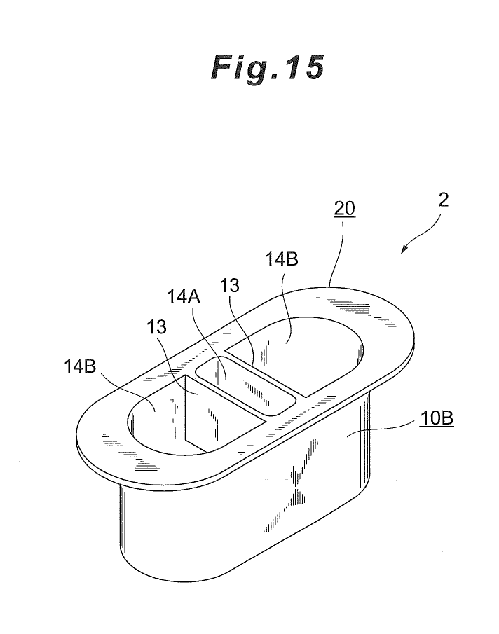

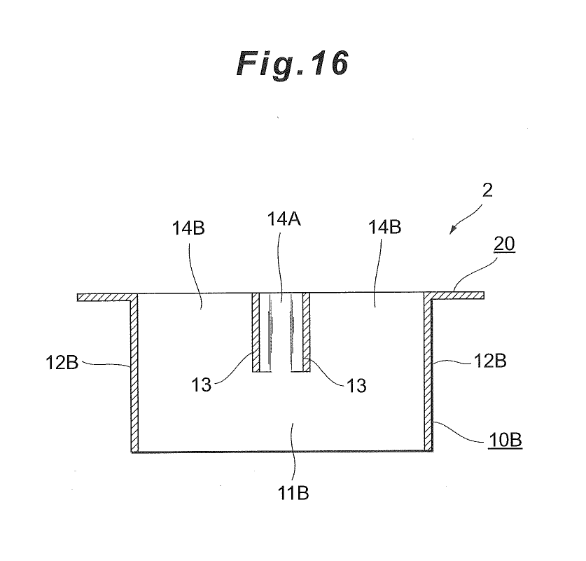

FIGS. 15 and 16 illustrate a trocar 2 according to a second embodiment of the present invention. The trocar 2 of the second embodiment differs from the trocar 1 of the first embodiment in that the outer flange 20 is integrally provided only on one end of the outer side of cylindrical portion 10B having the elliptical cross section, and no inner flange is provided; two side wall portions 11B and two arcuate portions 12B constituting the cylindrical portion 10B are provided perpendicular to the flange 20; and two partitioning wall portions 13 connecting the two side wall portions 11B are provided in spaced-apart relation, thereby partitioning the interior of the cylindrical portion 10B into three so that a total of three ports are formed, namely a central port 14A and two ports 14B on either side thereof. The central port 14A is narrower (smaller) than the ports 14B on either side and the four corners thereof are rounded.

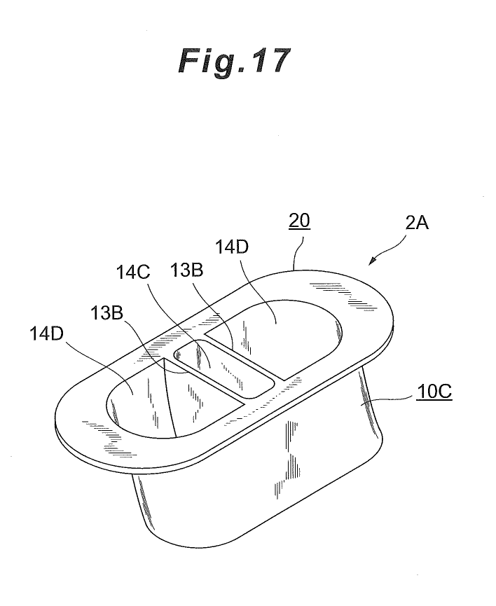

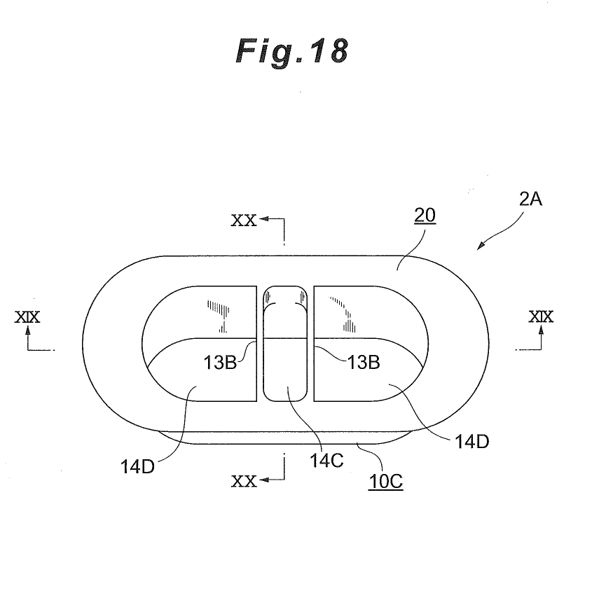

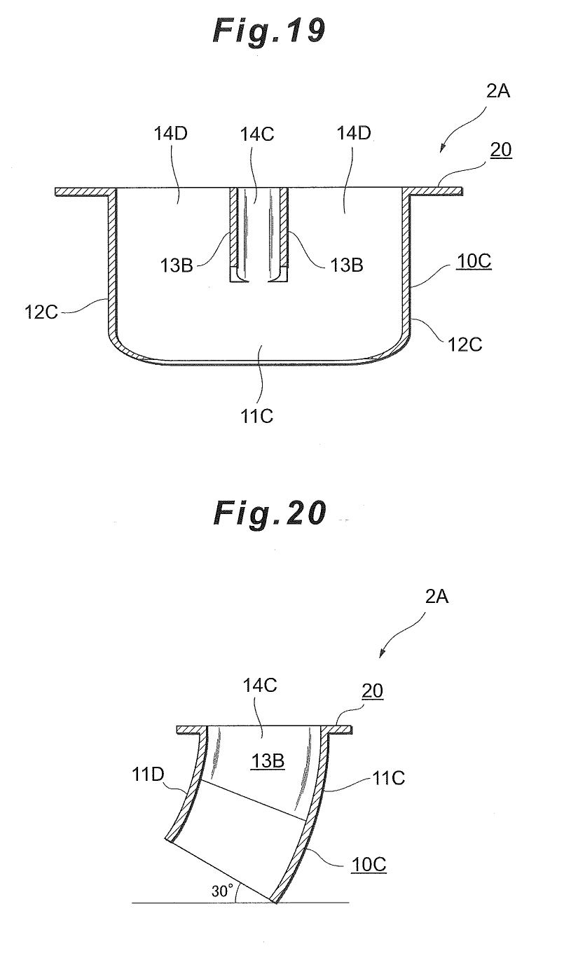

FIGS. 17 to 20 illustrate a modification of a trocar 2A according to the second embodiment. The trocar 2A of this modification differs from the trocar 2 of the second embodiment in that cylindrical portion 10C is curved smoothly in a direction connecting two side wall portions 11C and 11D. An angle of about 30° is formed between the upper end face on the outer side of the cylindrical portion 10C provided with the outer flange 20 and the lower end face on the inner side of the cylindrical portion 10C on the side opposite (see FIG. 20). Two arcuate portions 12C connecting both ends of the curved side wall portions 11C, 11D continuously and in arcuate fashion and two partitioning wall portions 13B partitioning the interior of the cylindrical portion 10C also are curved along the curvature of the cylindrical portion 10C. Since the cylindrical portion 10C is curved, ports 14C, 14D formed within the cylindrical portion 10C also are curved.

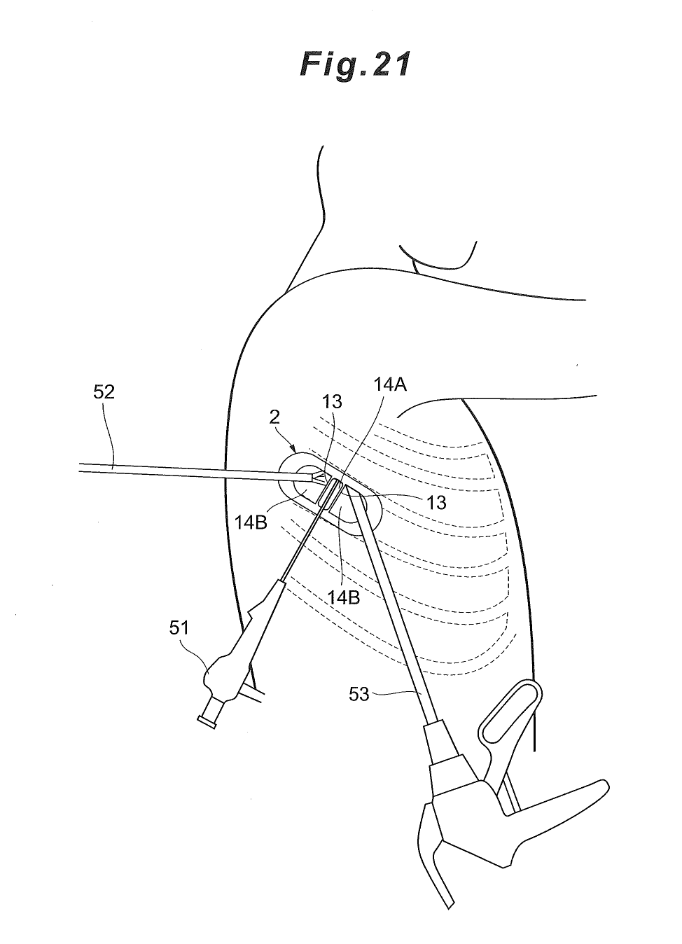

With reference to FIG. 21, since the trocars 2 and 2A of the second embodiment and modification thereof have three ports, multiple surgical instruments such as the scope 51, retractor 52 and stapler 53 can be inserted into the patient's body from respective one of these three ports 14 so that surgery and treatment can be carried out. When the trocar 2A having the curved cylindrical portion 10C is used, insertion of surgical instruments obliquely is facilitated. For example, since the scope 51 can be inserted obliquely, it is possible to widen the field of view for observation. The trocars 1, 1A, 1B, 2 and 2A of multiple types described above may be used properly in accordance with such factors as the area to undergo surgery and treatment and the scope thereof.

As many apparently widely different embodiments of the present invention can be made without departing from the spirit and scope thereof, it is to be understood that the invention is not limited to the specific embodiments thereof except as defined in the appended claims.

What is claimed is:

1. A trocar for thoracoscopic surgery for bringing into communication the inside and outside of the thoracic cavity which does not require that air tightness be maintained, the trocar being formed of a synthetic resin having such mechanical strength that the interior thereof will not collapse when the trocar is placed between the ribs, said trocar comprising:
a cylindrical portion with an elliptical cross section, and an outer flange and an inner flange provided on respective ones of both ends of said cylindrical portion;
said cylindrical portion having two side wall portions extending in the same direction, arcuate portions connecting the side wall portions at both ends thereof, and a partitioning wall portion at least partially partitioning the interior of said cylindrical portion between both side wall portions;
both side wall portions per se and both arcuate portions per se being formed in curved fashion in such a manner that outer surfaces thereof take on a concave shape from the outer flange to the inner flange.

2. The trocar according to claim 1, wherein said cylindrical portion is inclined in such a direction that dimensions thereof increase from the inner side to the outer side.

3. The trocar according to claim 1, wherein one end portion of the inner flange is formed tapering to a point.

4. The trocar according to claim 1, wherein the partitioning wall is provided extending in a direction from the outer side to the inner side of said cylindrical portion up to a point between both side wall portions.

5. The trocar according to claim 1, wherein the partitioning wall is provided extending from the outer side to the inner side of said cylindrical portion between both side wall portions.

6. The trocar according to claim 1, wherein the partitioning wall portion is constituted by a pair of projections each provided on the inner surface of a respective one of both side wall portions.

7. A port formation kit for thoracoscopic surgical instruments, comprising:
a trocar and a spacer;
said trocar including a cylindrical portion with an elliptical cross section, and an outer flange and an inner flange provided on respective ones of both ends of said cylindrical portion;
said cylindrical portion having two side wall portions extending in the same direction, arcuate portions connecting the side wall portions at both ends thereof, and at least one partitioning wall portion connecting both side wall portions;
said spacer having the shape of a U along the outer side of the cylindrical portion of said trocar.

8. The kit according to claim 7, wherein a hook is formed on a tip of said spacer.

9. The kit according to claim 7, including multiple spacers each of which has a different thickness.
